(19) United States
(12) Patent Application Publication
(10) Pub. No.: US 2025/0268843 A1
(43) Pub. Date: Aug. 28, 2025

(54) METHODS OF TREATMENT OF OSTEOARTHRITIS WITH TRANSDERMAL CANNABIDIOL GEL

(71) Applicant: Harmony Biosciences Management, Inc., Plymouth Meeting, PA (US)

(72) Inventors: Nancy Tich, Saint Paul, MN (US); John Messenheimer, Moncure, NC (US); Donna Gutterman, Raleigh, NC (US); Daniel Clauw, Ann Arbor, MI (US); Ted Smith, Tubac, AZ (US); James Griesser, Havertown, PA (US)

(73) Assignee: Harmony Biosciences Management, Inc., Plymouth Meeting, PA (US)

(21) Appl. No.: 19/007,534

(22) Filed: Jan. 1, 2025

Related U.S. Application Data
(63) Continuation of application No. 18/888,953, filed on Sep. 18, 2024, now abandoned.
Application No. 18/888,953 is a continuation of application No. 18/731,257, filed on Jun. 1, 2024, now abandoned.
Application No. 18/731,257 is a continuation of application No. 18/420,740, filed on Jan. 23, 2024, now abandoned.
Application No. 18/420,740 is a continuation of application No. 18/482,799, filed on Oct. 6, 2023, now abandoned.
Application No. 18/482,799 is a continuation of application No. 18/209,267, filed on Jun. 13, 2023, now abandoned.
Application No. 18/209,267 is a continuation of application No. 18/167,062, filed on Feb. 9, 2023, now abandoned.
Application No. 18/167,062 is a continuation of application No. 17/976,734, filed on Oct. 28, 2022, now abandoned.
Application No. 17/976,734 is a continuation of application No. 17/843,929, filed on Jun. 17, 2022, now abandoned.
Application No. 17/843,929 is a continuation of application No. 16/788,882, filed on Feb. 12, 2020, now abandoned.
Application No. 16/788,882 is a continuation of application No. PCT/IB2018/056090, filed on Aug. 13, 2018.
(60) Provisional application No. 62/661,733, filed on Apr. 24, 2018.
(60) Provisional application No. 62/545,468, filed on Aug. 14, 2017.

(51) Int. Cl. A61K 31/05; A61K 9/00; A61K 9/06; A61K 36/185; A61P 19/02

(52) U.S. Cl. CPC A61K 31/05; A61K 9/0014; A61K 9/06; A61K 36/185; A61P 19/02

(57) ABSTRACT

A pharmaceutical composition, and methods for using a pharmaceutical composition, for treating osteoarthritis in a subject in need thereof, the pharmaceutical composition including cannabidiol or a pharmaceutically acceptable salt thereof; and especially wherein administration of the cannabidiol is by a transdermal pharmaceutical gel composition. A method is disclosed for treating one or more symptoms of osteoarthritis in a patient. The method includes transdermally administering an effective amount of cannabidiol (CBD) to the patient wherein one or more symptoms of osteoarthritis are treated in the patient.

### CROSS REFERENCE TO RELATED APPLICATIONS

This application claims the benefit of U.S. provisional application Ser. No. 62/545,468 filed Aug. 14, 2017 and U.S. provisional application Ser. No. 62/661,733 filed Apr. 24, 2018. The entire disclosure of each of which is hereby incorporated herein by reference.

### FIELD OF THE TECHNOLOGY

The present disclosure generally relates to methods of treatment of osteoarthritis, and in particular, methods of treatment including transdermal administration of cannabidiol gel.

### BACKGROUND

The present disclosure relates to treatment of osteoarthritis using cannabidiol. Cannabidiol (CBD) is a cannabinoid having the formula:

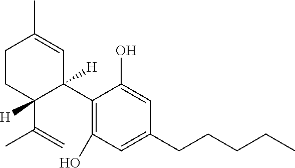 [SMILES: C=C(C)[C@@H]1CCC(C)=C[C@H]1c1c(O)cc(CCCCC)cc1O]

Osteoarthritis is a degenerative joint disease characterized by loss of cartilage and abnormal bone growth in joints of the patient. Symptoms include stiffness and pain, and can lead to decreased function or to disability. Osteoarthritis is estimated by the Centers for Disease Control and Prevention (CDC) to affect over 30 million adults in the United States.

Treatments for osteoarthritis known in the art include, among others, opioid pain medications. In recent years, abuse of opioid pain medications has become a major concern. Other existing treatments include nonsteroidal anti-inflammatory drugs (NSAIDs) such as COX-2 inhibitors. These treatments can have unfavorable side-effect profiles.

Many osteoarthritis patients using currently available medicines do not experience relief from pain and improved physical functioning, or cannot tolerate the medications due to side effects.

Evaluation of pain, stiffness and physical functioning can be used to determine whether a patient's osteoarthritis symptoms are improving. The Western Ontario & McMaster Universities Osteoarthritis Index (WOMAC) is a set of standardized questionnaires used by doctors and clinicians to evaluate the condition of patients with osteoarthritis of the knee and hip. It includes and evaluation of pain, stiffness, and physical functioning of the joints. The WOMAC consists of 24 items divided into three subscales and measures five items for pain, two for stiffness, and 17 for functional limitation.

### SUMMARY

Recent behavioral and electrophysiological studies have demonstrated that cannabinoids exert anti-nociceptive effects in rodent models of osteoarthritis. Cannabidiol (CBD) has been known to have antihyperalgesic effects through its interaction with TRPV1 receptors. CBD activates the adenosine receptor (A_{2A}), which has broad anti-inflammatory effects throughout the body. By blocking GPR55 signaling, which promotes osteoclast cell function, CBD can act to decrease bone resorption.

Cannabidiol (CBD) can provide treatment for osteoarthritis patients. The study disclosed herein shows that administration of a CBD transdermal gel to the skin of an osteoarthritic patient demonstrated improvement in a combined pain/function metric (composite responders). The study revealed particularly good results for male patients and for patients reporting a high baseline pain score at the outset of the study. Further, the study showed that treatment of patients with a lower dosage of cannabidiol, such as 250 mg daily, provided improved results over a dosage of 500 mg daily. The present disclosure provides a number of advantages. Transdermal delivery of CBD can have benefits over oral dosing because it allows the drug to be absorbed through the skin directly into the bloodstream. This avoids first-pass liver metabolism, potentially enabling lower dosage levels of active pharmaceutical ingredients with a higher bioavailability and improved safety profile. Transdermal delivery also avoids the gastrointestinal tract, lessening the opportunity for GI related adverse events and the potential degradation of CBD by gastric acid into THC, which can be associated with unwanted psychoactive effects. The treatment provided herein can reduce the frequency and severity of pain experienced by the patient. The treatment has limited side-effects. The treatment can be used by, for example, those who do not respond well to existing treatments, experience negative side-effects associated with such treatments, or, in the case of opioid treatments, where patients desire a non-opioid treatment.

In one aspect, a method for treating one or more symptoms of osteoarthritis in a patient is disclosed. The method includes transdermally administering an effective amount of cannabidiol (CBD) to the patient wherein one or more symptoms of osteoarthritis are treated in the patient.

In some embodiments, the effective amount is between 250 and 500 mg daily. The effective amount can be about 250 mg daily. The effective amount can be 500 mg daily. In some embodiments, the effective amount is 125 mg.

In some embodiments, the CBD is (-)-CBD. The CBD can be a synthetic CBD. The CBD can be a purified CBD. In some embodiments, the CBD is a botanically derived CBD.

The CBD can be formulated as a gel. In some embodiments, the CBD is formulated as a permeation-enhanced gel.

In some embodiments, the cannabidiol is within a pharmaceutical composition and the concentration of cannabidiol within the pharmaceutical composition is 3% to 5%. In some embodiments, the cannabidiol is within a pharmaceutical composition and the concentration of cannabidiol within the pharmaceutical composition is 4.2%.

Transdermally administering an effective amount of cannabidiol can include applying a gel suitable for transdermal application to the skin of the patient.

In some embodiments, the CBD is administered in a single daily dose. The CBD can be administered in two daily doses.

Transdermally administering an effective amount of CBD can reduce an intensity of an average worst knee pain score compared to a baseline. In some embodiments, the one or more symptom that is alleviated is knee pain. The one or more symptom that is alleviated can be pain and function. In some embodiments, alleviating one or more symptoms of osteoarthritis includes an improvement in physical function WOMAC score. Alleviating one or more symptoms of osteoarthritis can include an improvement in composite response analysis. In some embodiments, alleviating one or more symptoms of osteoarthritis can include an improvement in pain and function.

### BRIEF DESCRIPTION OF THE FIGURES

The invention can be more fully understood from the following detailed description in conjunction with the accompanying drawings, in which:

FIG. 1 shows a graph of mean weekly average worst knee pain score over time by treatment group.

FIG. 2 shows a chart of the mean reduction from baseline to week 12 in average worst knee pain scores

FIG. 3 shows a graph of mean percent change in weekly average worst knee pain score over time by treatment group.

FIG. 4 shows a chart of the composite responders at Last Observation Carried Forward (LOCF), where a composite responder is a patient who has a ≥30% reduction in pain and 20% response in physical function of WOMAC.

FIG. 5 shows a graph of median weekly average worst knee pain score over time by treatment group and sex (male).

FIG. 6 shows a chart of the composite responders at LOCF for males only.

FIG. 7 shows a graph of median weekly average worst knee pain score over time by treatment group and sex (female).

FIG. 8 shows a graph of median weekly average worst knee pain score over time by treatment group and baseline score for patients with baseline pain score greater than or equal to 7.

FIG. 9 shows a chart for the mean reduction from baseline to week 12 in average worst knee pain scores or patients with baseline pain score greater than or equal to 7.

FIG. 10 shows a graph of median weekly average worst knee pain score over time by treatment group and baseline score for patients with baseline pain score less than 7.

FIGS. 11A and 11B show charts for the composite response in patients with (11A) and without (11B) select medical histories.

### DETAILED DESCRIPTION

The present disclosure generally relates to administration of CBD medicament to the skin of a patient for transdermal permeation through and into the layers of the skin for delivery to the bloodstream of the patient.

As used herein, the term “cannabidiol” or “CBD” refers to cannabidiol; cannabidiol prodrugs; pharmaceutically acceptable derivatives of cannabidiol, including pharmaceutically acceptable salts of cannabidiol, cannabidiol prodrugs, and cannabidiol derivatives. CBD includes, 2-[3-methyl-6-(1-methylethenyl)-2-cyclohexen-1-yl]-5-pentyl-1,3-benzenediol as well as to pharmaceutically acceptable salts, solvates, metabolites (e.g., cutaneous metabolites), and metabolic precursors thereof. The synthesis of CBD is described, for example, in Petilka et al., Helv. Chim. Acta, 52:1102 (1969) and in Mechoulam et al., J. Am. Chem. Soc., 87:3273 (1965), which are hereby incorporated by reference.

As used herein, the term “treating” or “treatment” refers to mitigating, improving, relieving, or alleviating at least one symptom of a condition, disease or disorder in a subject, such as a human, or the improvement of an ascertainable measurement associated with a condition, disease or disorder.

As used herein, the term “transdermally administering” refers to contacting the CBD with the patient's or subject's skin under conditions effective for the CBD to penetrate the skin.

As used herein, the term “clinical efficacy” refers to the ability to produce a desired effect in humans as shown through a Food and Drug Administration (FDA), or any foreign counterparts, clinical trial.

As used herein, medicament includes any ointment, cream, solution, suspension, lotion, paste, gel, hydrogel, spray, foam, solid or oil which can be created or formed and used to administer CBD or a CBD prodrug) to a mammal, alone, or in conjunction with an bandage, patch, etc.

The CBD can be delivered transdermally to the patient by applying a CBD medicament topically. The CBD medicament can include inert diluents and carriers as well as other excipients, such as wetting agents, preservatives, and suspending and dispersing agents. In addition to these generally non-active components, topical formulations containing the CBD can further include additional active agents, particularly, active agents for the treatment of pain, discomfort, or otherwise for treating an illness of the patient. For example, the active materials can include analgesics, such as opiates and other analgesic active agents which operate on receptors other than CBD receptors.

The topical formulation can be applied directly to the skin and then optionally covered (e.g. with a bandage or gauze) to minimized the likelihood of disturbance. Alternatively, the topical formulation can be coated on the surface of a bandage, gauze, etc., or absorbed within the bandage, gauze, etc., such that the topical medicament is in direct contract with the patient's skin. The gel need not be administered proximate the arthritic joint or joints of the patient.

The effective amount as described herein can be a daily dosage amount of, for example, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, or about 700 mg. Any of the foregoing values can form a range; for example, a daily dose can be from about 125 mg to about 325 mg.

The effective amount as described herein can be a daily dosage amount of about 250 mg daily or about 500 mg daily. The dose can be administered as a single daily dose or the dose can be administered as a twice daily dose, for example, 125 mg twice daily or 250 mg twice daily.

The CBD can be in a gel form and can be pharmaceutically-produced as a clear, permeation-enhanced gel that is designed to provide controlled drug delivery transdermally with once-or twice-daily dosing. The CBD gel can between 1% (wt/wt) CBD to 7.5% (wt/wt) CBD. The CBD gel can have, for example, 4.2% (wt/wt) CBD or 7.5% (wt/wt) CBD). The CBD gel can be applied topically by the patient or caregiver to the patient's upper arm and shoulder, back, thigh, or any combination thereof.

The cannabidiol composition can include one or more of each of a permeation enhancer, solubilizing agent, a solubilizer, an antioxidant, a bulking gent, a thickening agent, or a pH modifier. Examples of the foregoing components are set for in the Handbook of Pharmaceutical Excipients, which is incorporated herein by reference. Permeation enhancers include: isostearic acid, octanoic acid, oleic acid, oleyl alcohol, lauryl alcohol, ethyl oleate, isopropyl myristate, butyl stearate, methyl laurate, diisopropyl adipate, glyceryl monolaurate, tetrahydrofurfuryl alcohol polyethylene glycol ether, polyethylene glycol, propylene glycol, 2-(2-ethoxyethoxy)ethanol, diethylene glycol monomethyl ether, alkylaryl ethers of polyethylene oxide, polyethylene oxide monomethyl ethers, polyethylene oxide dimethyl ethers, dimethyl sulfoxide, glycerol, ethyl acetate, acetoacetic ester, N-alkylpyrrolidone, and combinations thereof.

The CBD gel can include a solubilizing agent, a permeation enhancer, a solubilizer, antioxidant, bulking agent, thickening agent, and/or a pH modifier. The composition of the CBD gel can be, for example, a. cannabidiol present in an amount of about 0.1% to about 20% (wt/wt) of the composition; b. a lower alcohol having between 1 and 6 carbon atoms present in an amount of about 15% to about 95% (wt/wt) of the composition; c. a first penetration enhancer present in an amount of about 0.1% to about 20% (wt/wt) of the composition; and d. water in a quantity sufficient for the composition to total 100% (wt/wt). Other formulations of the CBD gel can be found in International Publication No. WO 2010/127033, the entire contents of which are incorporated herein by reference.

The CBD medicament can be stored within a bottle, tube, packet, sachet, pouch, or similar packaging. A sachet, and in particular a single-use sachet, can be provided such that the amount of medicament within the sachet is appropriate for a single use.

### EXEMPLIFICATION

Three hundred and twenty (320) patients aged 41 to 78 years of age with confirmed osteoarthritis of the knee were randomized in the double-blind, multi-center trial. Patients who completed the one-week washout and the seven-to-10-day baseline phase were randomized 1:1:1 to receive either 250 mg of CBD daily, 500 mg of CBD daily, or placebo, for 12 weeks. Enrolled patients had a mean worst knee pain score of 6.9 on a scale of 1 to 10 during baseline. The 250 mg and 500 mg doses were administered as a 4.2% (wt/wt) CBD gel. Patient disposition is shown in Table 1 and patient demographics for the safety analysis set is shown in Table 2.

TABLE 1
Patient Disposition

| | | | CBD Trans- | | CBD Trans- | |
|---|---|---|---|---|---|---|
| | | | dermal Gel | | dermal Gel | |
| | Placebo | | 250 mg/day | | 500 mg/day | |
| | n (%) | | n (%) | | n (%) | |
| Randomized | 107 | (100) | 107 | (100) | 106 | (100) |
| Safety Analysis Set | 106 | (99) | 107 | (100) | 106 | (100) |
| Efficacy Analysis Set | 103 | (96) | 106 | (99) | 105 | (99) |
| Completed Study | 71 | (66) | 84 | (79) | 81 | (76) |
| Discontinued Study | 36 | (34) | 23 | (22) | 25 | (24) |
| Due to AE | 8 | (8) | 8 | (8) | 4 | (4) |
| Withdrew Consent | 22 | (21) | 9 | (8) | 17 | (16) |
| Investigator Decision | 0 | (0) | 1 | (1) | 2 | (2) |
| Lost to Follow-Up | 4 | (4) | 2 | (2) | 0 | (0) |
| Other | 2 | (2) | 3 | (3) | 2 | (2) |

TABLE 2
Demographics (Safety Analysis Set)

| | | | CBD Trans- | | CBD Trans- | |
|---|---|---|---|---|---|---|
| | | | dermal Gel | | dermal Gel | |
| | Placebo | | 250 mg/day | | 500 mg/day | |
| | N = 106 | | N = 107 | | N = 106 | |
| Age, years (Mean) | 61.6 | | 61.6 | | 62.1 | |
| Sex, n (%) | | | | | | |
| Male | 54 | (51) | 55 | (51) | 48 | (45) |
| Female | 52 | (49) | 52 | (49) | 58 | (55) |
| Race, n (%) | | | | | | |
| White | 99 | (93) | 98 | (92) | 100 | (94) |
| Aboriginal | 2 | (2) | 1 | (1) | 2 | (2) |
| Asian | 0 | (0) | 4 | (4) | 3 | (3) |
| Other | 5 | (5) | 4 | (4) | 1 | (1) |

| | | | |
|---|---|---|---|
| Weight, kg (Mean) | 85.67 | 88.45 | 87.43 |
| BMI, kg/m² (Mean) | 30.13 | 30.84 | 30.57 |

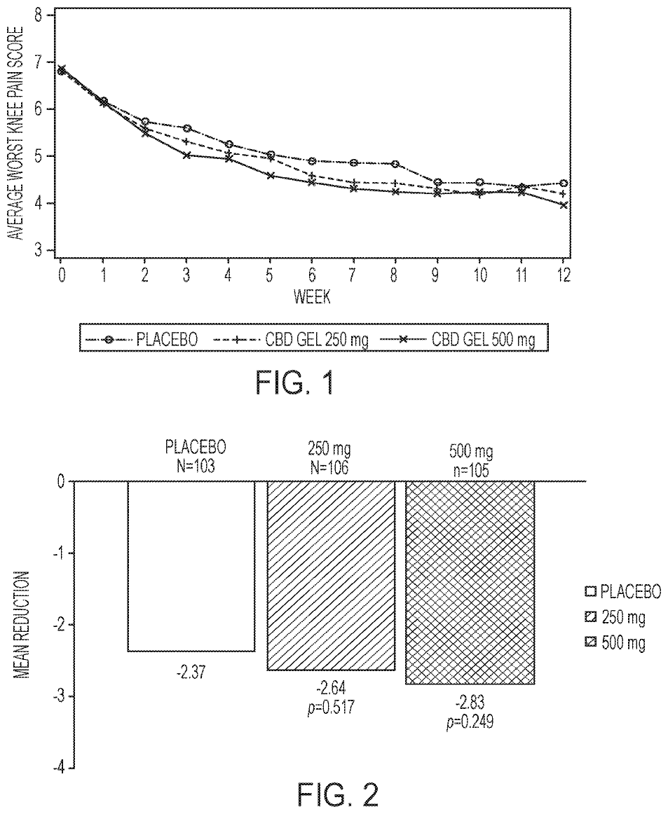

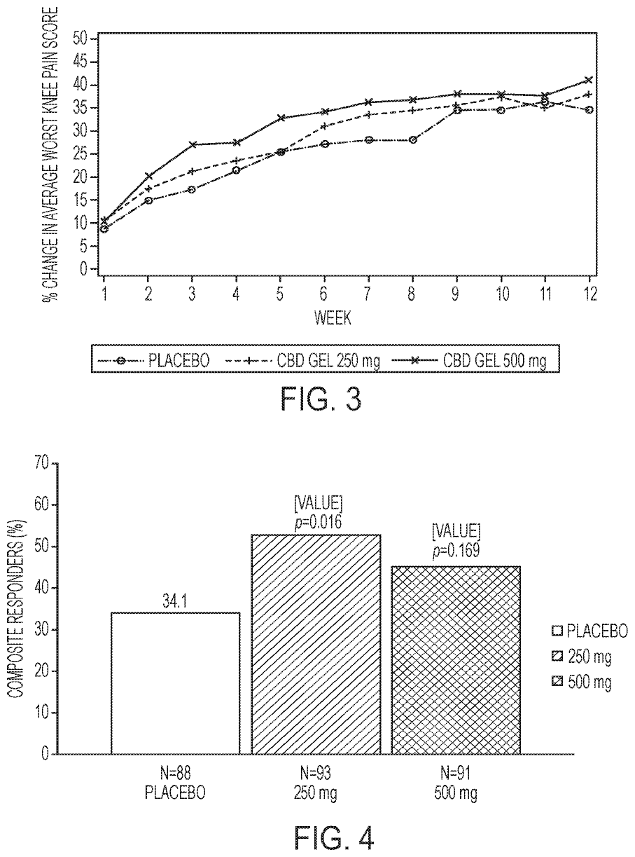

Across all participants, patients on 250 mg of CBD daily dosage achieved a 2.64 mean reduction from baseline in average worst knee pain scores at week 12; patients on 500 mg of CBD daily achieved a 2.83 mean reduction from baseline in average worst knee pain scores at week 12; and patients on placebo achieved a 2.37 mean reduction from baseline in average worst knee pain scores at week 12. These results were not statistically significant. FIGS. 1 and 2 show mean weekly average worst knee pain score over time by treatment group, and shows that pain decreased in both active groups and placebo. FIG. 3 shows the mean percent change in weekly average worst knee pain score corresponding to the results shown in FIG. 1. A mixed-model repeated measures (MMRM) model was used to test for differences between active treatment groups and placebo based on all weekly average worst knee pain scores from Baseline to Week 12.

Over an initial 7 day period, patients were instructed to end use of other osteoarthritic treatment such as NSAID or COX-2 inhibitor, but were permitted to use acetaminophen for rescue use. At this time, baseline mean worst knee pain was identified for each patient. During the study, rescue use of acetaminophen was permitted for both active and placebo study groups, and there was no difference among the groups in prevalence of rescue use.

Statistically significant results were achieved for the composite responder (i.e., the number and percent of patients with an average weekly improvement in worst pain score of ≥30% at last observation carried forward and a WOMAC physical function subscale reduction of ≥20% at last observation carried forward (LOCF)) analysis for 250 mg of CBD daily (p=0.016) as shown in FIG. 4. Fisher's Exact Test was used to test for differences in composite responders between each active treatment and placebo at LOCF. In the composite responder analysis, a positive response on the composite response analysis indicated a ≥30 percent reduction in worst average daily pain scores and a ≥20 percent improvement in WOMAC (Western Ontario & McMaster Universities Osteoarthritis Index) physical function score. Therefore, a positive composite response score demonstrates an improvement in both pain and function. It is believed that the composite responder score is more effective in distinguishing drug effect from placebo because the composite responder score includes both pain and function components.

A summary of the composite responder analyses at last observation is reported at Table 3. Table 3 shows that 60 out of 93 patients experienced a 20% or greater response in physical function WOMAC score after treatment with 250 mg daily of CBD in 4.2% CBD gel. Further, 53 out of 106 observed at least 30% reduction in pain, with a composite response of 49 out of 93 (52.7%) exhibiting the composite response.

TABLE 3
Summary of Composite Responder Analyses at Last Observation
Efficacy Patients

| | Placebo | 250 mg CBD | 500 mg CBD |
|---|---|---|---|
| Variable Category | (n = 103) | (n = 106) | (n = 105) |
| 30 Percent Reduction in Diary | | | |
| Pain | | | |
| Number of patients with | 103 | 106 | 105 |
| assessment | | | |
| Yes | 41 (39.8) | 53 (50.0) | 51 (48.6) |
| No | 62 (60.2) | 53 (50.0) | 54 (51.4) |
| 20 Percent Responders in Phys- | | | |
| ical Function WOMAC Score | | | |
| Number of patients with | 88 | 93 | 91 |
| assessment | | | |
| Yes | 45 (51.1) | 60 (64.5) | 54 (59.3) |
| No | 43 (48.9) | 33 (35.5) | 37 (40.7) |
| Composite Responder | | | |
| Number of patients with | 88 | 93 | 91 |
| assessment | | | |
| Yes | 30 (34.1) | 49 (52.7) | 41 (45.1) |
| No | 58 (65.9) | 44 (47.3) | 50 (54.9) |
| p-value | | 0.016 | 0.169 |
| (from a Fischer's Exact Test) | | | |

Statistically significant results were also recorded for the responder rate based on ≥30% reduction in worst pain severity at week 8 for 250 mg of CBD daily (p=0.037). A trend toward statistical significance was observed in other secondary endpoints.

In addition to demonstrating the value of cannabidiol for osteoarthritis, the present data also provide information regarding dosage levels. The 250 mg daily cannabidiol dosage shows better results that 500 mg daily in the composite assessment and in each component of the composite assessment. See FIG. 6. Further, the improving trend from 500 mg to 250 mg suggests that dosages lower than 250 mg can provide similar or greater benefit to patients. Dosage/weight analysis, discussed below, especially supports a reduced dosage (below 250 mg) in order to achieve the good results observed for low dosage/weight treatment.

Safety Data: The CBD gel was shown to be very well tolerated and the safety profile was consistent with previously released data from clinical trials. Table 4 provides a summary of adverse events.

TABLE 4
Summary of Adverse Events

| | | | CBD Trans- | | CBD Trans- | |
|---|---|---|---|---|---|---|
| | | | dermal Gel | | dermal Gel | |
| | Placebo | | 250 mg/day | | 500 mg/day | |
| | N = 106 | | N = 107 | | N = 106 | |
| | n (%) | | n (%) | | n (%) | |
| Patients with at least 1 | 45 | (42.5) | 53 | (49.5) | 53 | (50) |
| TEAE | | | | | | |
| Application site dryness | 1 | (1) | 5 | (5) | 3 | (3) |
| Application site reaction | 1 | (1) | 3 | (3) | 0 | |
| Application site pain | 0 | | 3 | (3) | 0 | |
| Headache | 4 | (4) | 6 | (6) | 2 | (2) |

| | | | | |
|---|---|---|---|---|
| Dizziness | 0 | 0 | 3 | (3) |

Pharmacokinetic Data: The dose media plasma concentrations for the 500 mg and 250 mg daily CBD doses were dose proportional, with the 500 mg dose levels being approximately two times the plasma concentration of the 250 mg dose.

The data obtained in the study described in Example 1 was also analyzed with respect to gender. Men on 250 mg of CBD daily achieved a 1.65 mean reduction from baseline in average worst knee pain scores at week 4 compared to a 1.19 mean reduction from baseline in men on placebo (p=0.156); a 2.30 mean reduction from baseline in average worst knee pain scores at week 8 compared to a 1.56 mean reduction from baseline in men on placebo (p=0.066); and a 2.68 mean reduction from baseline in average worst knee pain scores at week 12 compared to a 1.70 mean reduction from baseline in men on placebo (p=0.049).

TABLE 5
Worst Knee Pain Score by Week for Male Patients
MALE PATIENTS

| | placebo | | 250 mg | | 500 mg | |
|---|---|---|---|---|---|---|
| baseline | n = 54 | 6.9 | n = 55 | 6.77 | n = 48 | 6.85 |
| wk 4 | | 5.61 | | 5.05 | | 5.33 |
| pvalue | | | | 0.156 | | 0.323 |
| % reduction | | 17% | | 24% | | 22% |
| wk 8 | | 5.24 | | 4.47 | | 4.73 |
| pvalue | | | | 0.066 | | 0.474 |
| % reduction | | 23% | | 34% | | 30% |
| wk 12 | n = 34 | 5.21 | n = 47 | 4.16 | n = 38 | 4.44 |
| pvalue | | | | 0.049 | | 0.193 |
| % reduction | | 25% | | 39% | | 33% |

Composite Responders

| | | | |
|---|---|---|---|
| | 27% | 60% | 41% |
| pvalue | | 0.003 | 0.174 |

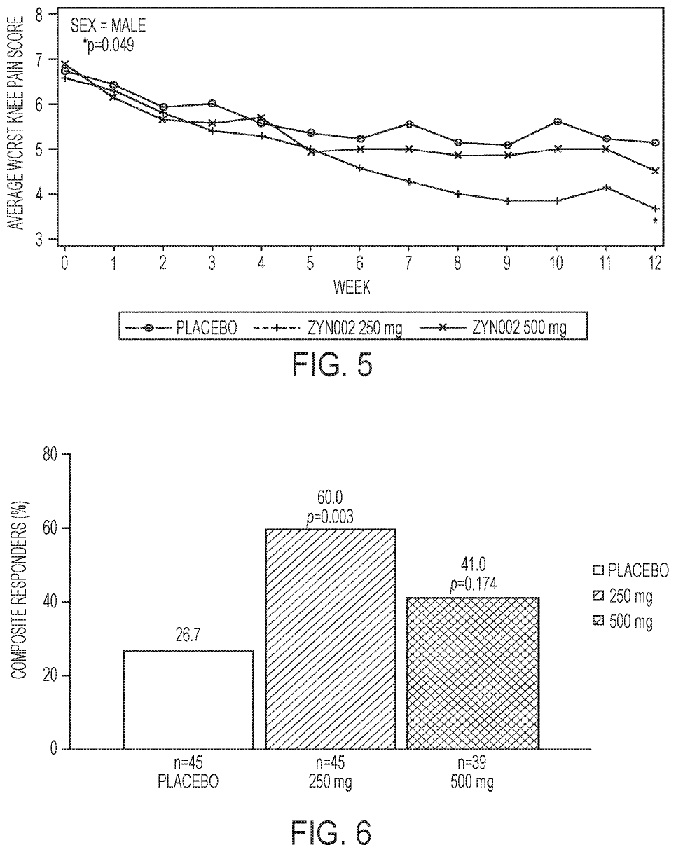

FIG. 5 shows median weekly average worst knee pain score over time for men, and shows that men experienced a significant reduction in mean knee pain score as over placebo for both 250 mg CBD and 500 mg CBD, with lowest mean pain scores reported for patients receiving 250 mg CBD at the study end point. FIG. 6 shows the composite responders at LOCF for males only.

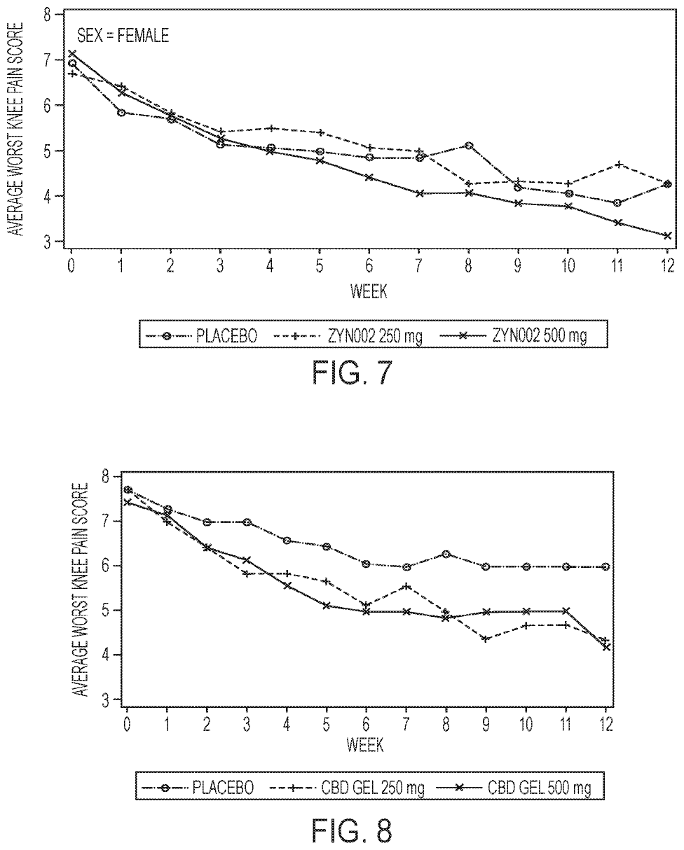

While female patients showed a reduction in pain, as shown in FIG. 6, the female placebo group demonstrated a greater reduction in pain. Females did not achieve significance in the endpoint. FIG. 7 shows median weekly average worst knee pain score over time for female patients, showing that pain decreased for both active and placebo arms over the course of the study, but with no significance at end of study.

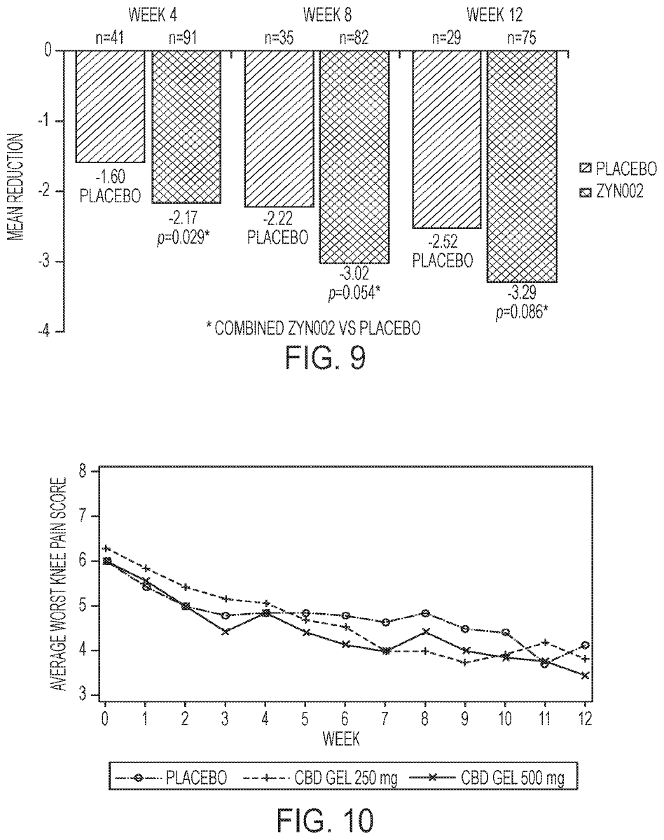

The data obtained in the study was also analyzed with respect to response at different levels of pain severity and showed greater effectiveness in patients with a higher baseline pain score. The mean baseline worst knee pain score was 6.9. In an evaluation of patients with a baseline pain score of ≥7, there were 101 patients on CBD and 48 patients in the placebo arm in this baseline range within this group. Patients on CBD achieved a 2.17 mean reduction from baseline in average worst knee pain scores at week 4 compared to a 1.6 mean reduction from baseline in patients on placebo (p=0.029); a 3.02 mean reduction from baseline in average worst knee pain scores at week 8 compared to a 2.22 mean reduction from baseline in patients on placebo (p=0.054); and a 3.29 mean reduction from baseline in average worst knee pain scores at week 12 compared to a 2.52 mean reduction from baseline in patients on placebo (p=0.086). See FIG. 9.

TABLE 6
Worst Knee Pain Score by Week for
Patients with ≥7 Mean Baseline Pain Score.
≥7

| | placebo | | CBD | |
|---|---|---|---|---|
| baseline | n = 48 | 7.82 | n = 101 | 7.7 |
| wk4 | | 6.21 | | 5.45 |
| pvalue | | | | 0.029 |
| % reduction | | 20% | | 28% |
| wk8 | | 5.7 | | 4.61 |
| pvalue | | | | 0.054 |
| % reduction | | 28% | | 40% |
| wk12 | n = 29 | 5.43 | n = 75 | 4.34 |
| pvalue | | 0.086 | | |
| % reduction | | 32% | | 43% |

Composite Responders

| | | |
|---|---|---|
| | 33% | 51% |
| pvalue | | 0.083 |

FIG. 8 shows that knee pain score for patients having a baseline knee pain score greater than or equal to 7 was lowered for both 250 mg CBD and 500 mg CBD to a greater degree than for placebo. FIG. 9 shows the mean reduction from baseline to week 12 in average worst knee pain scores for patients having a baseline knee pain score greater than or equal to 7. The data in FIG. 9 is presented as the placebo group versus the combined CBD transdermal gel (i.e., both dosage groups combined). By comparison, FIG. 10 shows the corresponding data for patients having a baseline knee pain score less than 7. For this group, pain reduction over placebo was not statistically significant.

The foregoing results in Table 5 and Table 6 can also be compared with the overall patent totals are reported in Table 7, demonstrating that results for male patients and for patients with a baseline pain greater than or equal to 7 show greater pain reduction than for the overall study population, and relatively less reduction associated with placebo.

TABLE 7
Worst Knee Pain Score by Week for Entire Study.
TOTAL PATIENTS

| | placebo | | 250 mg | | 500 mg | |
|---|---|---|---|---|---|---|
| baseline | n = 103 | 6.81 | n = 106 | 6.8 | n = 105 | 6.87 |
| wk 4 | | 5.26 | | 5.09 | | 4.96 |
| pvalue | | | | 0.432 | | 0.067 |
| % reduction | | 22% | | 24% | | 27% |
| wk 8 | | 4.85 | | 4.43 | | 4.27 |
| pvalue | | | | 0.193 | | 0.21 |
| % reduction | | 28% | | 34% | | 37% |
| wk 12 | n = 67 | 4.45 | n = 76 | 4.23 | n = 75 | 3.99 |
| pvalue | | | | 0.517 | | 0.249 |
| % reduction | | 35% | | 38% | | 41% |

Composite Responders

| | | | |
|---|---|---|---|
| | 34% | 53% | 45% |
| pvalue | | 0.016 | 0.169 |

The variability in baseline pain scores affects efficacy. Table 8 shows the composite response as a function of baseline pain standard deviation.

TABLE 8
Composite Response as a Function of Baseline Pain Standard Deviation

| | | CBD Transdermal Gel | CBD Transdermal Gel |
|---|---|---|---|
| | Placebo | 250 mg/day | 500 mg/day |
| Lower 33% Baseline Pain | 27% (n = 10/37) | 50% (n = 17/34) | 52% (n = 15/29) |
| Standard Deviation | | (p = 0.055) | (p = 0.046) |
| Middle 33% Baseline Pain | 40% (n = 10/25) | 57% (n = 20/35) | 26% (n = 7/27) |
| Standard Deviation | | (NS) | (NS) |
| Upper 33% Baseline Pain | 39% (n = 10/26) | 50% (n = 12/24) | 54% (n = 19/35) |
| Standard Deviation | | (NS) | (NS) |

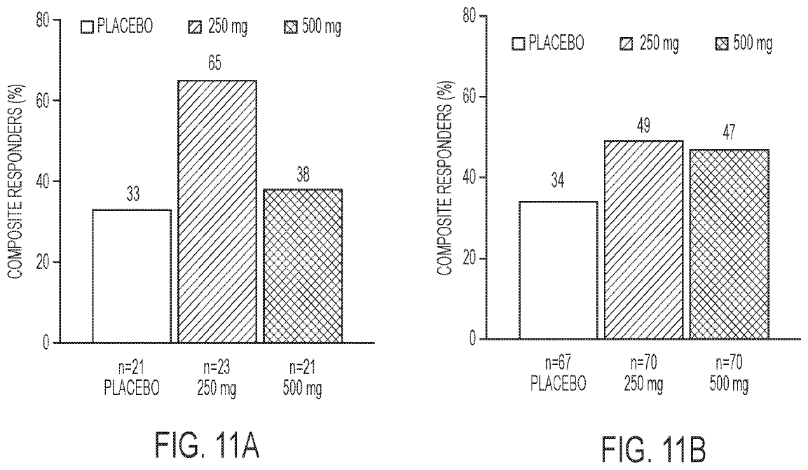

The composite response was also analyzed in patients with an without select medical histories. Select medical histories consists of central neuropathic pain, fibromyalgia, depression, mood change, dysphoria, fatigue and/or insomnia. FIGS. 11A and 11B is a chart detailing the composite response in patients with and without select medical histories.

The study data was also analyzed with regard to the active dose per weight of the patient (i.e., dosage/weight in mg CBD/kg weight of patient). For this analysis, the patient population was divided into three groups: 0-25 percentile, 25-75 percentile, and 75-100 percentile, by dose/weight. Table 9 is a summary of composite responder analysis at last observation Dose/Weight Quartile 0-25%. Table 10 is a summary of composite responder analysis at last observation dose/weight quartile 25-75%. Table 11 is a summary of composite responder analysis at last observation dose/weight quartile 75-100%.

TABLE 9

| | Placebo | CBD gel 250 mg |
|---|---|---|
| Variable Category | (N = 103) | (N = 52) |
| 30 Percent Reduction in Diary Pain | | |
| Number of Patients with assessment | 103 | 52 |
| Yes | 41 (39.8) | 29 (55.8) |
| No | 62 (60.2) | 23 (44.2) |
| 20 Percent Responders in Physical | | |
| Function WOMAC Score | | |
| Number of Patients with assessment | 88 | 46 |
| Yes | 45 (51.1) | 32 (69.6) |
| No | 43 (48.9) | 14 (30.4) |
| Composite Responders | | |
| Number of Patients with assessment | 88 | 46 |
| Yes | 30 (34.1) | 27 (58.7) |
| No | 58 (65.9) | 19 (41.3) |
| p-value | | 0.010 |

TABLE 10

| | | CBD gel | CBD gel |
|---|---|---|---|
| | Placebo | 250 mg | 500 mg |
| Variable Category | (N = 103) | (N = 54) | (N = 52) |
| 30 Percent Reduction in Diary | | | |
| Pain | | | |
| Number of Patients with | 103 | 54 | 52 |
| assessment | | | |
| Yes | 41 (39.8) | 24 (44.4) | 26 (50.0) |
| No | 62 (60.2) | 30 (55.6) | 26 (50.0) |
| 20 Percent Responders in Phys- | | | |
| ical Function WOMAC Score | | | |
| Number of Patients with | 88 | 47 | 45 |
| assessment | | | |
| Yes | 45 (51.1) | 28 (59.6) | 26 (57.8) |
| No | 43 (48.9) | 19 (40.4) | 19 (42.2) |
| Composite Responders | | | |
| Number of Patients with | 88 | 47 | 45 |
| assessment | | | |
| Yes | 30 (34.1) | 22 (46.8) | 22 (48.9) |
| No | 58 (65.9) | 25 (53.2) | 23 (51.1) |
| p-value | | 0.194 | 0.133 |

TABLE 11

| | Placebo | CBD gel 500 mg |
|---|---|---|
| Variable Category | (N = 103) | (N = 53) |
| 30 Percent Reduction in Diary Pain | | |
| Number of Patients with assessment | 103 | 53 |
| Yes | 41 (39.8) | 25 (47.2) |
| No | 62 (60.2) | 28 (52.8) |
| 20 Percent Responders in Physical | | |
| Function WOMAC Score | | |
| Number of Patients with assessment | 88 | 46 |
| Yes | 45 (51.1) | 28 (60.9) |
| No | 43 (48.9) | 18 (39.1) |
| Composite Responders | | |
| Number of Patients with assessment | 88 | 46 |
| Yes | 30 (34.1) | 19 (41.3) |
| No | 58 (65.9) | 27 (58.7) |
| p-value | | 0.453 |

This analysis shows that patients within the 0-25 percentile group exhibited the greatest percentage of composite responders, with 27 of 46 (58.7%) exhibiting both the 30 percent reduction in pain and 20 percent response in physical function WOMAC score. Fully 69.6% of patients in this group demonstrated 20 percent response in physical function WOMAC score. By comparison, fewer than half of the patients in the 25-75 percentile groups exhibited the composite response (46.8% for the 250 mg CBD and 48.9% for the 500 mg CBD subsets). For the 75-100 percentile group, only 41.3% exhibited the composite response. Thus, the analysis of dosage/weight showed that a lower dosage/weight was more effective. Further, the analysis permits comparison of two groups of 250 mg of CBD patients: those receiving a lower dosage/weight (0-25 percentile) and a higher dosage/weight (25-75 percentile). This comparison shows that within the 250 mg of CBD group, a lower dosage/weight demonstrated a better composite responder score. This trend indicates that a lower dosage amount than 250 mg can be beneficial to patients. Lower dosages below 250 mg will permit more patients to fall within the low dosage/weight range of active demonstrated herein to yield favorable results.

### Summary of Results

Neither the 250 mg nor the 500 mg dose of the CBD transdermal gel groups demonstrated statistically significant separation from placebo in terms of mean reduction from baseline to week 12 in average worst knee pain scores. Analysis revealed that a significant separation of CBD transdermal gel relative to placebo was observed for those with a baseline page score≥7. Analysis highlighted that females exhibited a greater placebo response than males.

A significantly greater number of patients receiving 250 mg CBD transdermal gel daily (52.7%) were composite responders compared to those receiving placebo (34.1%). Analysis revealed that the difference from placebo for both 250 mg and 500 mg groups widened among those who had the lease amount of variability in baseline pain scores.

All publications and references cited herein are expressly incorporated herein by reference in their entirety.

1. A method for treating one or more symptoms of osteoarthritis in a patient, the method comprising
transdermally administering an effective amount of cannabidiol (CBD) to the patient wherein one or more symptoms of osteoarthritis are treated in the patient.

2-22. (canceled)
